(19) United States
(12) Patent Application Publication
(10) Pub. No.: US 2018/0338723 A1
(43) Pub. Date: Nov. 29, 2018

(54) APPARATUS FOR MONITORING SEATED INDIVIDUALS

(71) Applicant: Holly Heilner, Harbeson, DE (US)

(72) Inventor: Holly Heilner, Harbeson, DE (US)

(21) Appl. No.: 15/864,081

(22) Filed: Jan. 8, 2018

Related U.S. Application Data
(60) Provisional application No. 62/511,781, filed on May 26, 2017.

(51) Int. Cl. A61B 5/00; A61B 5/11

(52) U.S. Cl. CPC A61B 5/6891; A61B 5/1115; A61B 5/1126; A61B 5/746; A61B 5/1117

(57) ABSTRACT

A system for monitoring a seated or supine individual includes a system base and a first hinged arm and a second hinged arm on the system base. The first hinged arm and the second hinged arm each have a range of motion from an engaged position to a free position. The system includes an alarm module positioned to recognize a positional change of the first hinged arm and/or the second hinged arm. The alarm module is configured to produce an alarm when one of first hinged arm and second hinged arm are moved from the first position, and is further configured to turn off an alarm when each of first hinged arm and second hinged arm is returned to the first position, and/or in response to instructions from a supervising individual. The alarm is suitable to alert a supervising individual, e.g., nurse, doctor, guardian, etc.

### CROSS REFERENCE TO RELATED APPLICATION(S)

This application claims the benefit of U.S. Provisional Patent Application No. 62/511,781, filed May 26, 2017, which is incorporated by reference as if disclosed herein in its entirety.

### BACKGROUND

According to the Joint Commission, a non-profit organization that accredits more than 21,000 health care organizations and programs in the United States, the average cost of a patient fall with injury is $14,000.00. In addition, each patient fall with injury adds an average of 6.3 days to the patient's hospital stay. Falls can cause major injuries—even death. Patients in a hospital, short term rehab, and long term care facilities are many times at very high risk for falls due to their weakened physical condition or impairment from pain or sedative medications.

The policy at most facilities is that patients with a certain fall score, which usually consists of 80% of the patients, must be accompanied to and from the bathroom and the staff member must stay inside the bathroom with the patient. Because of census, i.e., the population, at most facilities, staff is unable to do this. Also, many patients refuse to allow staff to go into the bathroom with them because they want their privacy and they instead go into the bathroom alone. Patients often promise the staff they will pull the cord when they are finished and wait for staff to assist them. This does not happen because patients think they can do it alone and do not pull the cord for assistance prior to getting up from the toilet, which often results in patient falls.

Bathrooms have many hard surfaces. Approximately 50% of all falls in a facility are toileting related (not all are inside the bathroom). In addition, many patients in these facilities are on anticoagulant medicines. If a patient on anticoagulant medication falls and hits their head, they could suffer a brain bleed and die.

There are current technologies used by facilities alone or in combination to attempt to address this problem. Most facilities have the call bell with a cord for the patient to pull when they need assistance. Many facilities have hand rails or grab bars installed. Some facilities use a sensor pad that sits between the rim of the toilet and the toilet seat. When the patient stands up, the alarm sounds—but only after the patient gets up and/or falls. Even using all of the above devices, the shortcomings are that none of the above prevents the patient from falling off the toilet. Calling for assistance depends on the patient's decision to pull the cord. And, the alarm does not go off until after the patient begins to rise or falls off the toilet.

The drawback is that it is cumbersome for staff to carry to the bathroom. (In the same way raised toilet seat devices are carried to the bathrooms and placed over the toilets.) However, having portable ones allows them to be utilized where necessary. But once it is placed in the bathroom, it can stay there for the patient's entire stay. The portable device could have wheels placed on it; however, this is a double edged sword, because if it is not anchored properly to the wall or wheel locks used, the device could become a fall hazard. There are pros and cons to have a permanent or portable device.

### SUMMARY

A system according to the disclosed subject matter are directed to a system for monitoring a seated or supine individual including a frame including a first side frame and a second side frame opposing the first side frame. In some embodiments, a first radially rotatable arm on the first side frame. In some embodiments, a second radially rotatable arm on the second side frame. In some embodiments, the first radially rotatable arm and the second radially rotatable arm are positioned transversely. In some embodiments, the first radially rotatable arm and the second radially rotatable arm each have a range of motion from a first position in a substantially horizontal plane to a second position in a substantially vertical plane. In some embodiments, the first side frame, the second side frame, the first radially rotatable arm, and the second radially rotatable arm define a recess between them. In some embodiments, a mounting mechanism configured to secure the frame to a surface.

In some embodiments, an alarm module is positioned to recognize a positional change of the first radially rotatable arm or the second radially rotatable arm. In some embodiments, alarm module produces an alarm when one of the first radially rotatable arm and the second radially rotatable arm are moved from the first position. In some embodiments, the alarm module turns off an alarm when each of the first radially rotatable arm and the second radially rotatable arm is returned to the first position. In some embodiments, the alarm is any alarm suitable to alert a supervising individual, e.g., nurse, doctor, guardian, etc.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a schematic diagram of a system for monitoring a seated or supine individual according to some embodiments of the present disclosure;

FIG. 2A is a schematic diagram of a system for monitoring a seated or supine individual according to some embodiments of the present disclosure;

FIG. 2B is a schematic diagram of a system for monitoring a seated or supine individual according to some embodiments of the present disclosure;

FIG. 2C is a schematic diagram of a system for monitoring a seated or supine individual according to some embodiments of the present disclosure; and

FIG. 3 is a chart of a method of using a system for monitoring a seated or supine individual according to some embodiments of the present disclosure.

### DETAILED DESCRIPTION

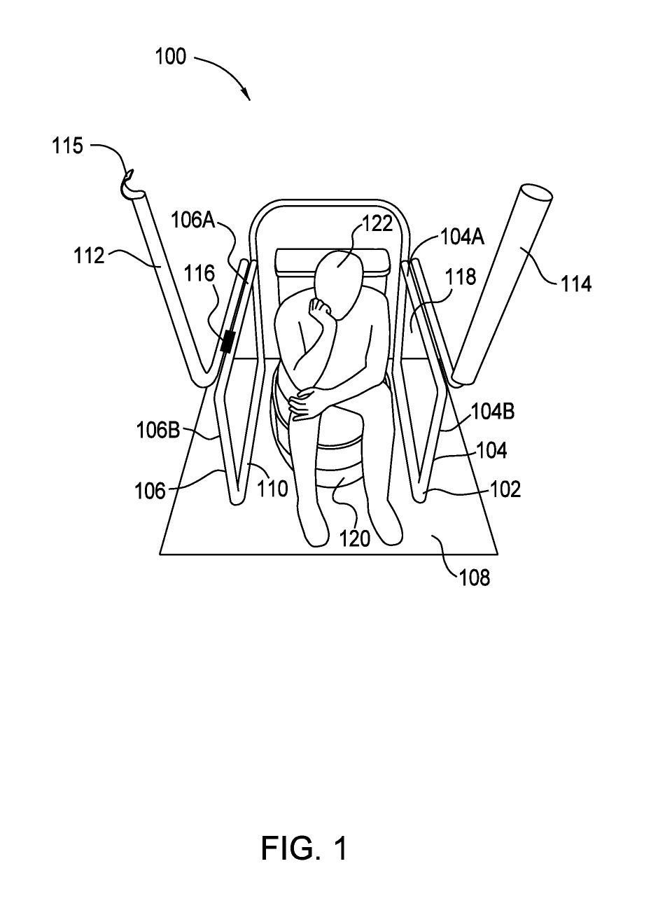

Referring now to FIG. 1, aspects of the disclosed subject matter include a system 100 for monitoring a seated or supine individual. In some embodiments, system 100 includes a system base 102. In some embodiments, system base 102 includes a first side frame 104. In some embodiments, system base 102 includes a second side frame 106. In some embodiments, first side frame 104 is positioned opposing second side frame 106. In some embodiments, first side frame 104 includes a first horizontal frame member 104A. In some embodiments, first side frame 104 includes a first vertical frame member 104B. In some embodiments, second side frame 106 includes a second horizontal frame member 106A. In some embodiments, second side frame 106 includes a second vertical frame member 106B.

In some embodiments, system base 102 is secured to a surface 108. In some embodiments, surface 108 includes a floor, wall, ceiling, seating apparatus, frame for a seating apparatus, or a combination thereof. In some embodiments, the seating apparatus includes chairs, bed, commodes, toilets, stools, stretchers, etc., or a combination thereof. In some embodiments, the frame for a seating apparatus includes a bed frame, stretcher frame, etc., or a combination thereof. In some embodiments, system base 102 is secured to surface 108 at a mounting point via mounting mechanism 110. In some embodiments, system base 102 is secured reversibly to surface 108. In some embodiments, system base 102 is secured permanently to surface 108. In some embodiments, system 100 is free-standing. In some embodiments, system base 102 is portable. In some embodiments, system base 102 includes wheels configured to aid in the portability of system 100 from one location to another.

In some embodiments, system 100 includes a first arm 112 and a second arm 114. In some embodiments, first arm 112 and second arm 114 are connected to system base 102. In some embodiments, first arm 112 and second arm 114 are parallel. In some embodiments, first arm 112 and second arm 114 are transverse. In some embodiments, first arm 112 and/or second arm 114 are substantially straight. In some embodiments, first arm 112 and/or second arm 114 have a curved or bent shape. In some embodiments, at least one of first arm 112 and second arm 114 include padding. In some embodiments, at least one of first arm 112 and second arm 114 include a stabilizing mechanism 115 configured to receive a portion of system base 102. In some embodiments, stabilizing mechanism 115 configured to receive a portion of one of first arm 112 and second arm 114. In some embodiments, stabilizing mechanism 115 includes a cavity. In some embodiments, first arm 112 and/or second arm 114 are secured and/or connected to an existing seating apparatus or frame for an existing seating apparatus.

First arm 112 and second arm 114 are configured to be movable from a first position to a second position. In some embodiments, first arm 112 and/or second arm 114 are slidable. In some embodiments, first arm 112 and/or second arm 114 are rotatable. In some embodiments, first arm 112 and/or second arm 114 are radially rotatable. In some embodiments, the first position is in a substantially horizontal plane. In some embodiments, the first position is an “engaged” position, such that the arm provides a barrier to user movement out of system base 102. In some embodiments, the engaged position is substantially perpendicular to system base 102. In some embodiments, stabilizing mechanism 115 engages with a portion of system base 102 when in the engaged position. In some embodiments, the second position is a “free” position, such that the arm presents less restriction to user movement out of system base 102 than when in the first position. In some embodiments, the second position is in a substantially vertical plane. In some embodiments, the second position is past vertical, i.e., the relevant arm moves from a substantially horizontal position to and past a vertical position. The past vertical free position prevents the relevant arm from falling back to the engaged position once released by a user. In some embodiments, movement between the second position and the first position is reversible, in that the arm is free to move in at least one direction at all times, and is unencumbered by a locking mechanism, e.g., a latch. In some embodiments, first arm 112 and/or second arm 114 include a hinge to enable movement from the first position to the second position.

In some embodiments, system 100 includes an alarm module 116. Alarm module 116 is configured and/or positioned to recognize a positional change of first arm 112 and/or second arm 114. In some embodiments, alarm module 116 is configured to produce an alarm when one of first arm 112 and second arm 114 are moved from the first position. In some embodiments, alarm module 116 is configured to turn off the alarm in response to instructions from a supervising individual. In some embodiments, alarm module 116 is configured to turn off an alarm and/or reset the alarm module when each of first arm 112 and second arm 114 is returned to the first position. In some embodiments, the alarm can only be turned off by returning first arm 112 and second arm 114 to the first position. The alarm is any alarm suitable to alert a supervising individual, e.g., nurse, doctor, guardian, etc. In some embodiments, the alarm is a sound, light, vibration, generated signal, etc., or a combination thereof. In some embodiments, alarm module 116 is configured to silence an alarm in response to instructions from the supervising individual. As used herein, a silenced alarm still acknowledges an alarm condition is active, however the alarm itself becomes less obtrusive, e.g., an audiovisual alarm becomes just a visual alarm, a sound alarm becomes a visual alarm, etc. In some embodiments, the alarm is silenced via a silencing mechanism, e.g., a button, switch, signal generated via user interaction with a computer application, etc. In some embodiments, the silencing mechanism is positioned remotely from the seated or supine individual so that that individual cannot silence the alarm themselves. In some embodiments, the alarm is transmitted to a location or device remote from system 100, e.g., a sound or light is emitted in another room. In some embodiments, the alarm is transmitted via a wired connection. In some embodiments, the alarm is transmitted via a wireless connection. In some embodiments, system 100 includes a status indicator which indicates the alarm status of alarm module 116. In some embodiments, the status indicator is light or combination of lights configured to display one or more colors of light. In some embodiments, the status indicator is configured to provide an indication of whether alarm module 116 is activated, deactivated, malfunctioning, on, i.e., in an alarm condition, silenced, off, etc.

In some embodiments, system 100 is sized and configured to comply with applicable codes and regulations. In some embodiments, first side frame 104 and second side frame 106 are spaced apart by about 20 inches to about 30 inches. In some embodiments, first horizontal frame member 104A and/or the second horizontal frame member 106A are positioned at a height of about 30 inches to about 37 inches. In some embodiments, first vertical frame member 104B and/or second vertical frame member 106B are about 30 inches to about 37 inches in height. In some embodiments, first arm 112 and the second arm 114 are positioned at a height of about 30 inches to about 37 inches when in the first position.

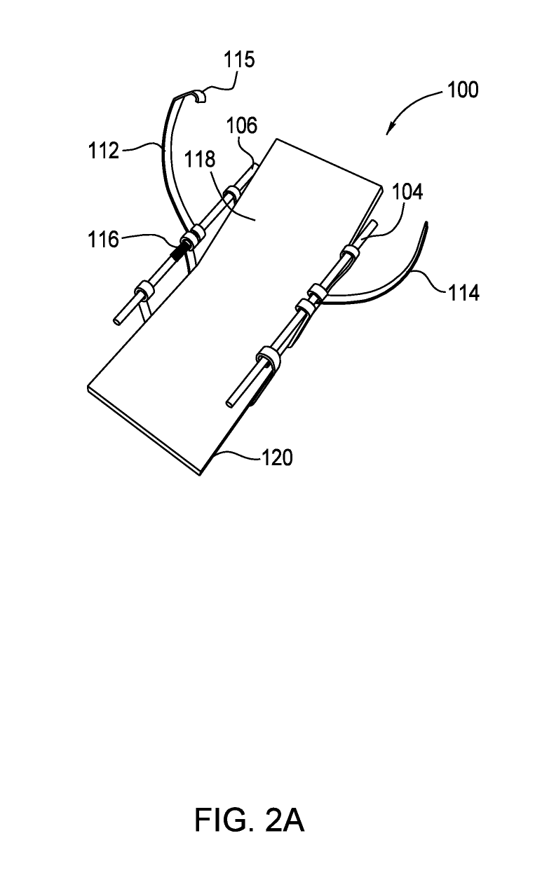

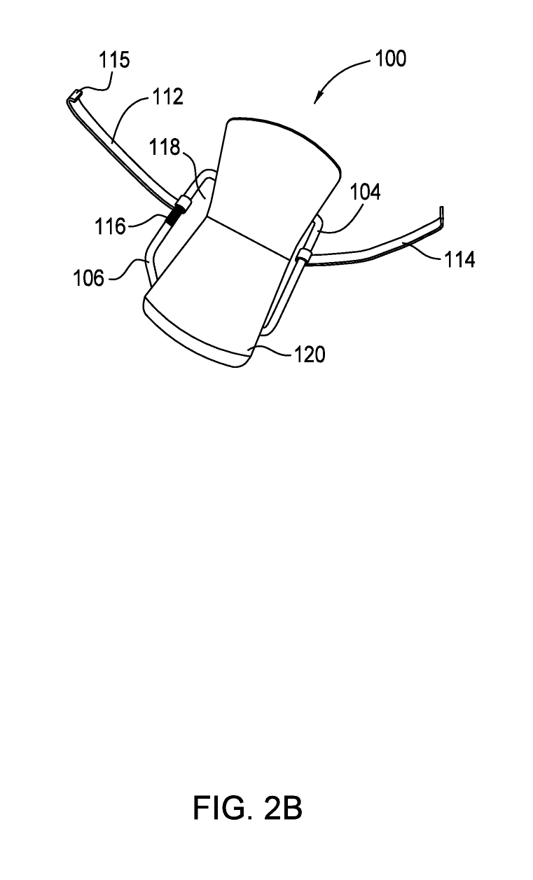

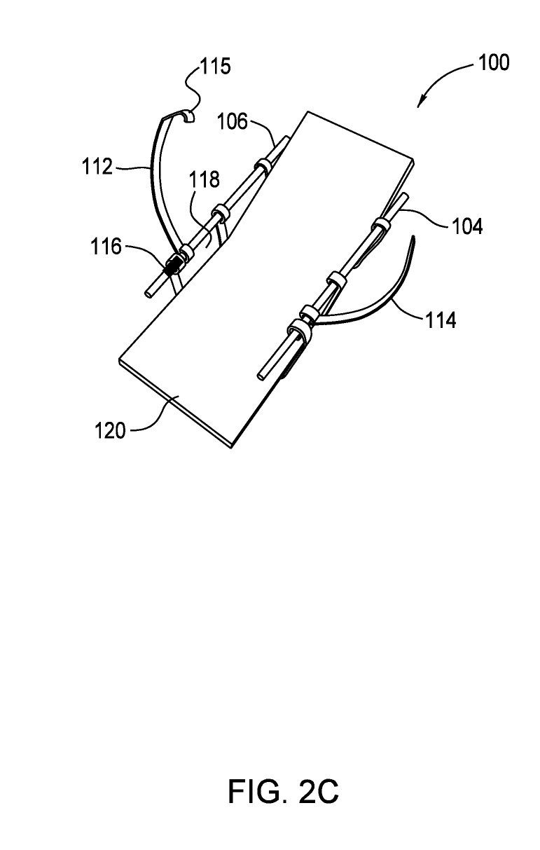

In some embodiments, first arm 112, second arm 114, and system base 102 and/or seating apparatus 120 define a recess 118 there between. In some embodiments, recess 118 has dimensions sized to accommodate a seating apparatus 120 and an individual 122 seated upon the seating apparatus. FIG. 1 portrays an embodiment of system 100 positioned to monitor and assist an individual 122 on a chair, toilet, etc. Referring now to FIGS. 2A-2B, system 100 can also be positioned on a seating apparatus 200, e.g., a stretcher (FIG. 2A) or a chair (FIG. 2B). Referring now to FIG. 2C, system 100 can also be positioned on an end 210 of stretcher 210 in the event an individual 122 were to inch off the foot of the stretcher.

In some embodiments, system base 102, first arm 112, second arm 114, or combinations thereof are configured to support the weight of individual 122. In some embodiments, first arm 112 and second 114 are composed of lightweight material for easy transportation and movement of system 100 and its components. In some embodiments, recess 118 is sized to provide sufficient space for individual 122 to comfortably sit and/or lie down within the recess, as well as perform certain actions related to seating apparatus 120. For example, referring again to FIG. 1, in embodiments where seating apparatus 120 is a toilet, recess 118 is sized to accommodate individual 122 while seated on the toilet, as well as while leaning forward while cleaning themselves. In some embodiments, first arm 112 and second arm 114 are movable along system base 102 to alter the size of recess 118, so that the recess can be sized to accommodate individuals 122 of different sizes and/or allow for tailored ranges of movement based on the individual and system application.

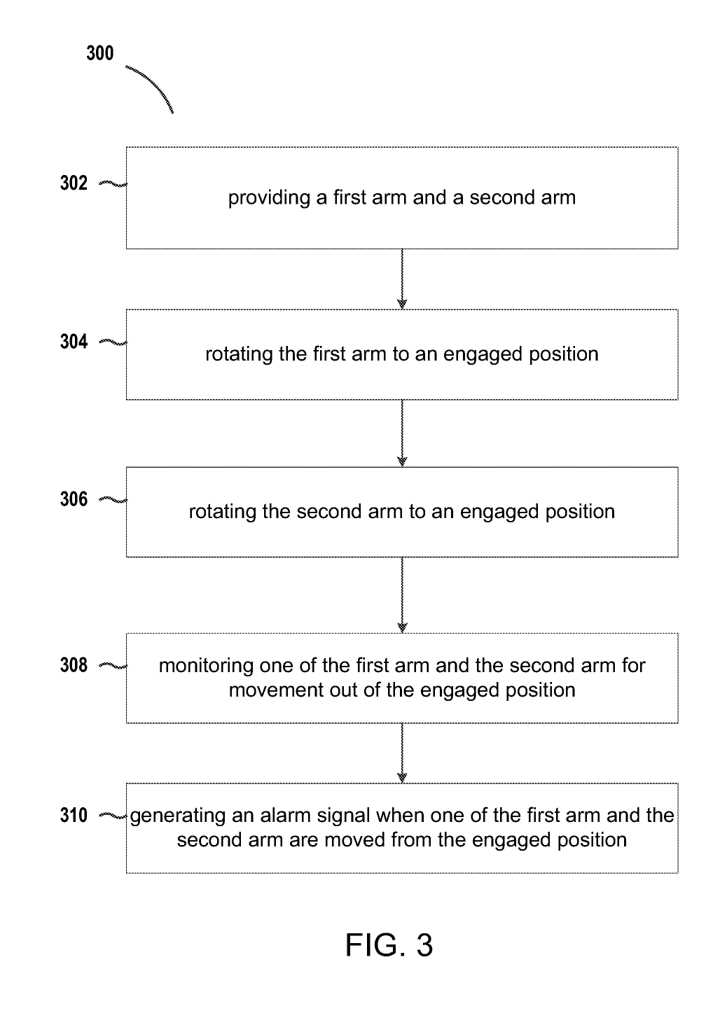

Referring now to FIG. 3, aspects of the disclosed subject matter include a method 300 of monitoring an individual in a seated or supine position. At 302, a first arm and a second arm are provided. As discussed above, the first and second arms are movable, e.g., rotatable. At 304, the first arm is moved to an engaged position. At 306, the second arm is moved to an engaged position. As discussed above, in some embodiments, the movable arms are reversibly moved from a past-vertical position. At 308, the first arm and the second arm are monitored for movement out of the engaged position. At 310, an alarm signal is generated when one of the first arm and the second arm are moved from the engaged position.

Methods and systems according the disclosed subject matter offer advantages over known technology. The system base and arms provide non-invasive support for users upon seating apparatus, preventing accidents and the pain, discomfort, recuperation, and financial costs often associated with those accidents. While the system of the present disclosure does provide supports which impede an individual's ability to accidentally fall from a seating apparatus, the system is not configured to confine that individual, and is not considered a restraint. For users, therefore, the system is empowering rather than restrictive, granting the user more freedom to operate independently and providing much wanted privacy in the bathroom. This is also possible because the arms and alarm module of the system provide peace of mind to supervising individuals such as caregivers, nurses, and doctors, as they know they will be alerted before the user rises from the seating apparatus. In addition, because the alarm rings when an individual starts to lift the first arm, the system provides supervising individuals time to react and enter the bathroom or patient room before the patient actually rises, while the patient is still in the seated or supine position, unlike other sensors that alarm after the patient has left the seat.

Although the disclosed subject matter has been described and illustrated with respect to embodiments thereof, it should be understood by those skilled in the art that features of the disclosed embodiments can be combined, rearranged, etc., to produce additional embodiments within the scope of the invention, and that various other changes, omissions, and additions may be made therein and thereto, without parting from the spirit and scope of the present invention.

What is claimed is:

1. A system for monitoring a seated or supine individual comprising:
a system base;
a first hinged arm on the system base;
a second hinged arm on the system base and transverse the first hinged arm; and
an alarm module positioned to recognize a positional change of the first hinged arm or the second hinged arm;
wherein the first hinged arm and the second hinged arm each have a range of motion from an engaged position to a free position.

2. The system according to claim 1, further comprising mounting points configured to secure the system to a surface.

3. The system according to claim 1, wherein the alarm module is configured to produce an alarm when one of the first hinged arm and the second hinged arm are moved from the engaged position.

4. The system according to claim 3, wherein the alarm module is configured to turn off the alarm in response to instructions from a supervising individual.

5. The system according to claim 3, wherein the alarm module is configured to turn off the alarm when each of the first hinged arm and the second hinged arm is returned to the engaged position.

6. The system according to claim 1, wherein the first hinged arm includes a cavity shaped to receive the base in the engaged position.

7. The system according to claim 1, wherein the engaged position is substantially perpendicular to the base.

8. The system according to claim 1, wherein the system base, the first hinged arm, and the second hinged arm define a recess there between.

9. The system according to claim 1, wherein the system base includes a wall, frame, or a combination thereof.

10. A system for monitoring a seated or supine individual comprising:
a frame including a first side frame and a second side frame opposing the first side frame;
a mounting mechanism configured to secure the frame to a surface;
a first radially rotatable arm on the first side frame;
a second radially rotatable arm on the second side frame; and
an alarm module positioned to recognize a positional change of the first radially rotatable arm or the second radially rotatable arm;
wherein the first side frame, the second side frame, the first radially rotatable arm, and the second radially rotatable arm define a recess there between, and the first radially rotatable arm and the second radially rotatable arm each have a range of motion from a first position in a substantially horizontal plane to a second position in a substantially vertical plane, and
the recess has dimensions sized to accommodate a seating apparatus and an individual seated upon the seating apparatus.

11. The system according to claim 10, wherein the first radially rotatable arm and the second radially rotatable arm are positioned at a height of about 30 inches to about 37 inches when in the first position.

12. The system according to claim 10, wherein the first side frame includes a first horizontal frame member and the second side frame includes a second horizontal frame member, the first horizontal frame member and the second horizontal frame member are positioned at a height of about 30 inches to about 37 inches, and the first side frame and the second side frame are spaced apart by about 20 inches to about 30 inches.

13. The system according to claim 10, wherein the alarm module is configured to produce an alarm when one of the first radially rotatable arm and the second radially rotatable arm are moved from the first position.

14. The system according to claim 13, wherein the alarm module is configured to turn off the alarm in response to instructions from a supervising individual.

15. The system according to claim 14, wherein the alarm module is configured to turn off the alarm when each of the first radially rotatable arm and the second radially rotatable arm is returned to the first position.

16. The system according to claim 10, wherein the first radially rotatable arm includes a stabilizing mechanism configured to receive the frame.

17. The system according to claim 10, wherein movement between the second position and the first position is reversible.

18. The system according to claim 10, wherein the second position is past vertical.

19. A method of monitoring an individual in a seated or supine position comprising:
providing a first radially rotatable arm and a second radially rotatable arm;
rotating the first radially rotatable arm to an engaged position;
rotating the second radially rotatable arm to an engaged position;
monitoring one of the first radially rotatable arm and the second radially rotatable arm for movement out of the engaged position; and
generating an alarm signal when one of the first radially rotatable arm and the second radially rotatable arm are moved from the engaged position.

20. The method according to claim 19, wherein the steps of rotating the first radially rotatable arm and the second radially rotatable arm to an engaged position further comprise:
reversibly rotating the radially rotatable arms from a past-vertical position.
